(19) United States
(12) Patent Application Publication
(10) Pub. No.: US 2026/0055358 A1
(43) Pub. Date: Feb. 26, 2026

(54) PHENOTYPIC AND BIOLOGICAL ASSESSMENT OF MICROBES

(71) Applicant: Inscripta, Inc., Pleasanton, CA (US)

(72) Inventors: Glenn Patrick Hein, San Lorenzo, CA (US); Christopher Michael Rath, Oakland, CA (US); Theodore M. Tarasow, Orinda, CA (US)

(21) Appl. No.: 19/105,536

(22) PCT Filed: Aug. 22, 2023
(86) PCT No.: PCT/US23/30816

§ 371 Date: Feb. 21, 2025

Related U.S. Application Data
(60) Provisional application No. 63/400,304, filed on Aug. 23, 2022.
(60) Provisional application No. 63/406,480, filed on Sep. 14, 2022.

(51) Int. Cl. C12M 1/36; C12M 1/34; G06V 20/69; G16C 20/30; G16C 20/70

(52) U.S. Cl. CPC C12M 41/48; C12M 41/36; G06V 20/698; G16C 20/30; G16C 20/70

(57) ABSTRACT

The present disclosure provides technologies for predicting a phenotype of a microbial cell using machine learning models trained using high-content imaging data (HCI). Also provided are methods of engineering a microbial cell to possess a phenotype of interest. Example phenotypes include the production of a target compound or biomolecule of interest. The provided technologies are useful for the efficient biomanufacturing of target compounds.

### CROSS-REFERENCE TO RELATED APPLICATIONS

This application claims priority to U.S. Provisional Application No. 63/400,304, filed Aug. 23, 2022; and U.S. Provisional Application No. 63/406,480, filed Sep. 14, 2022, the entire contents of each of which are incorporated herein by reference.

### BACKGROUND

The following discussion is merely provided to aid the reader in understanding the disclosure and is not admitted to describe or constitute prior art thereto.

While microbial cell-based biomanufacturing continues to be developed and improved, the development of new strains or improvement to production from existing strains is limited by numerous factors. Current methods often involve extensive and specific knowledge of the biology of a given microbial cell. Moreover, there are a large number of factors affecting the yield of a target biomolecule produced by a microbial cell, which can make exhaustive experimental testing infeasible. In addition, use of reductionist approaches (e.g., testing the effect(s) of one strain modification at a time), attractive for its simplicity in a complex environment, often hides effects that can be seen only in combinations of such modifications. Further, assessing the fitness and productivity of a large number of candidate microbial cells to be used for bioproduction can be costly at scale.

The development and expansion of computational biology approaches has greatly advanced biomanufacturing. Secondary or alternative computational approaches may improve the optimization of biomanufacturing in certain technology areas. Unlike traditional metabolic modeling, which is based on mass and energy balances derived from reconstructed metabolic networks, data-driven algorithms, such as machine learning (ML) approaches, make predictions by extracting patterns from experimentally generated data. ML-based computational strategies function by deriving patterns from data without the need for mechanistic understanding.

### SUMMARY

The present disclosure provides systems and methods of determining, detecting, and predicting the phenotype of microorganisms (e.g., yeast) grown in fermentation tanks or bioreactors based on physical characteristics of the microorganism. The physical characteristics can be assessed by staining the microorganism and assessing various features that have been stained or are otherwise measureable in, for example, an image of the microorganism. These disclosed systems and methods represent an improvement over the current state of the art by providing a mechanism to determine, detect, and predict phenotype in a quick and non-invasive way, whereas prior art techniques required laborious, time-consuming, and expensive techniques (e.g., genotyping, measuring titers of a desired compound, measuring growth/survival over time).

In one aspect, the present disclosure provides a method for determining or predicting a phenotype of a microbial cell, comprising: (a) culturing the microbial cell; (b) obtaining at least one high-content image of the microbial cell; (c) executing a computer-based model that was trained with image features of known microbial cell phenotypes; and (d) generating, with a processor, a determination or prediction of the phenotype of the microbial cell. In some implementations, the microbial cell is a yeast cell. In some implementations, the known microbial cell phenotypes associated with the image feature are selected from titer of a compound of interest that is produced by the microbial cell, knock-out of a gene of interest, expression of a gene of interest, microbial fitness, a stress response, or a combination thereof. In some implementations, the computer-based model is a deep learning model or a logistic model. In some implementations, the at least one high-content image is a fluorescent microscopy image. In some implementations, the fluorescent microscopy image comprises at least one fluorescent channel. In some implementations, the at least one high-content image is a bright field image. In some implementations, the method further comprises, before obtaining at least one high-content image of the microbial cell, fixing the microbial cell with a fixing agent. In some implementations, fixing comprises allowing the microbial cell to adhere to a surface of a container containing the microbial cells.

In one aspect, the present disclosure provides a method for determining or predicting titer of a compound of interest by a microbial cell, comprising: executing a computer-based model to analyze at least one high content image of a microbial cell that produces a compound of interest, wherein the computer-based model is trained with image features associated with known microbial cell titers; and generating, with a processor, a determination or prediction of the titer of the compound of interest being produced by the microbial cell. In some implementations, the compound of interest is a terpene or terpenoid. In some implementations, the compound of interest is selected from the group consisting of bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene. In some implementations, the microbial cell is a yeast cell. In some implementations, the computer-based model is a deep learning model or a logistic model. In some implementations, the at least one high-content image is a fluorescent microscopy image. In some implementations, the fluorescent microscopy image comprises at least one fluorescent channel. In some implementations, the at least one high-content image is a bright field image. In some implementations, the method further comprises executing a second computer-based model trained with image features associated with known microbial cell phenotypes to determine or predict a second phenotype selected from knock-out of a gene of interest, expression of a gene of interest, microbial fitness, a stress response, or a combination thereof.

In one aspect, the present disclosure provides a method of engineering a microbial cell to have a desired phenotype, comprising: (a) generating, in silico, at least one design candidate microbial cell incorporating at least one genetic feature associated with a desired phenotype; (b) engineering the at least one design candidate microbial cell; (c) culturing the at least one design candidate microbial cell; and (d) determining the phenotype of the at least one design candidate microbial cell using a high-content imaging (HCI)-based model. In some implementations, determining the phenotype of the at least one design candidate microbial cell comprises (i) obtaining at least one high-content image of the at least one design candidate microbial cell; (ii) executing a computer-based model that was trained with image features associated with the at least one phenotypic measure; and (iv) generating with a processor a prediction of the phenotype of the microbial cell. In some implementations, the HCI-based model is trained with a data set, comprising: i) at least one input variable representing the at least one genetic feature, and ii) at least one measured phenotypic performance output variable representing at least one phenotypic measurement associated with the genetic feature, wherein the at least one phenotypic measurement corresponds to a HCI image feature. In some implementations, the method further comprises, before determining the phenotype of the at least one design candidate microbial cell, fixing the at least one design candidate microbial cell with a fixing agent. In some implementations, fixing comprises allowing the at least one design candidate microbial cell to adhere to a surface of a container containing the at least one design candidate microbial cell.

In one aspect, the present disclosure provides a bioreactor or fermentation monitoring system, comprising: (a) a tank for culturing a population of microbial cells, (b) a camera for obtaining high content images of the population of microbial cells in the tank, (c) a processing system connected to the camera such that the high content images obtained by the camera is used to predict a phenotype or function of individual cells within the population of microbial cells while the population of cells is being cultured.

In one aspect, the present disclosure provides a computer-implemented method for predicting a phenotype of a microbial cell, comprising: (a) populating a predictive machine learning model with a training data set, comprising: i) at least one input variable representing at least one genetic alteration that has been introduced into a microbial cell, and ii) at least one measured phenotypic performance output variable representing at least one phenotypic measurement associated with the introduced genetic alteration, wherein the at least one phenotypic measurement comprises a high-content image (HCI) image feature related to the phenotypic measurement; (b) generating, in silico, a pool of design candidate microbial cells incorporating the at least one genetic alteration; and (c) utilizing the predictive machine learning model to predict the expected phenotypic measurement of members of the pool of design candidate microbial cells that comprise a combination of genetic alterations selected from (a) that are uncharacterized for improving phenotypic performance at the time of carrying out (c); wherein the predicted expected phenotypic measurement is selected from the group consisting of titer, growth properties, omics data, and production of a product of interest. In some implementations, the product of interest is selected from the group consisting of: a small molecule, enzyme, protein, peptide, amino acid, organic acid, synthetic compound, fuel, alcohol, primary extracellular metabolite, secondary extracellular metabolite, intracellular component molecule, and combinations thereof. In some implementations, the predictive machine learning model is stored and executed on a computer system comprising a processor and a non-transitory computer-readable medium (CRM). In some implementations, the processor is coupled to the non-transitory CRM.

In one aspect, the present disclosure provides a method of staining a microbial cell, comprising: (a) culturing the microbial cell; (b) fixing the microbial cell with a fixing agent (e.g., adhering the cells to a plate); (c) staining the microbial cell with at least one staining agent; (d) imaging the microbial cell, thereby generating an image of the microbial cell; and (e) predicting a phenotype of the microbial cell based on the image of the microbial cell. In some implementations, the microbial cell is a yeast cell. In some implementations, the fixing agent is formaldehyde. In some implementations, the at least one staining agent is a fluorescent staining agent. In some implementations, staining comprises staining the microbial cell with at least two staining agents. In some implementations, the at least two staining agents are fluorescent conjugates of Phalloidin and Concanavalin A. In some implementations, imaging the microbial cell comprises acquiring a high-content image of the microbial cell. In some implementations, predicting a phenotype of the microbial cell comprises (i) executing a computer-based model that was trained with image features of known microbial cell phenotypes; and (ii) generating, with a processor, a determination or prediction of the phenotype of the microbial cell.

In one aspect, the present disclosure provides a method of predicting a phenotype of a microbial cell, comprising: (a) culturing the microbial cell; (b) fixing the microbial cell with a fixing agent (e.g., adhering the cells to a plate); (c) staining the microbial cell with at least one staining agent; (d) imaging the microbial cell, thereby generating an image of the microbial cell; and (e) predicting a phenotype of the microbial cell based on the image of the microbial cell. In some implementations, the microbial cell is a yeast cell. In some implementations, the fixing agent is formaldehyde. In some implementations, the at least one staining agent is a fluorescent staining agent. In some implementations, staining comprises staining the microbial cell with at least two staining agents. In some implementations, the at least two staining agents are fluorescent conjugates of Phalloidin and Concanavalin A. In some implementations, imaging the microbial cell comprises acquiring a high-content image of the microbial cell. In some implementations, predicting a phenotype of the microbial cell comprises (i) executing a computer-based model that was trained with image features of known microbial cell phenotypes; and (ii) generating, with a processor, a determination or prediction of the phenotype of the microbial cell. In some implementations, after generating the determination or prediction, the determination or prediction is used to train the computer-based model.

For the purposes of the disclosed method and any of the foregoing aspects or implementations, the microbial cell may be prokaryotic or eukaryotic. In some implementations, the microbial cell is prokaryotic and can be selected from Escherichia coli (E. coli), an Acinetobacter species, a Pseudomonas species, a Streptomyces species, a Bacillus species, and a Mycobacterium species. In some implementations, the microbial cell is eukaryotic and can be selected from a yeast, a filamentous fungus, an algae, and an amoeba. In some implementations, the filamentous fungus is selected from an Aspergillus species and a Trichoderma species. In some implementations, the amoeba is Dictyostelium discoideum. In some implementations, the algae is selected from Botryococcus braunii, Chlorella sp., Crypthecodinium cohnii, Cylindrotheca sp., Nitzschia sp., Phaeodactylum tricornutum, Schizochytrium sp., and Tetraselmis suecia. In some implementations, the yeast is Saccharomyces cerevisiae (S. cerevisiae), Pichia pastoris, or Kluyveromyces marxianus. In some implementations, the yeast is an oleaginous yeast.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### DETAILED DESCRIPTION

Implementations according to the present disclosure will be described more fully hereinafter. Aspects of the disclosure may, however, be embodied in different forms and should not be construed as limited to the implementations set forth herein. Rather, these implementations are provided so that this disclosure will be thorough and complete, and will fully convey the scope of the invention to those skilled in the art. The terminology used in the description herein is for the purpose of describing particular implementations only and is not intended to be limiting. The practice of the present technology may employ techniques of molecular biology, microbiology, chemical engineering, and cell biology, which are within the skill of the art.

Unless the context indicates otherwise, it is specifically intended that the various features described herein can be used in any combination. Moreover, the disclosure also contemplates that in some implementations, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

### Definitions

As used in the description of the invention and the appended claims, the singular forms “a,” “an” and “the” are intended to include the plural forms as well, unless the context clearly indicates otherwise.

As used herein, “about” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself. Thus, by way of example, “about 10” should be understood to mean “10” and a range no larger than “9-11”.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

As used herein, an image is a representation of an object captured by an imaging device, such as a camera.

As used herein, the term “image feature” generally refers to any data that can be measured or extracted from an image. An image feature may be related to the structure or function of an imaged object. For example, an image feature of an image of a biological cell may be related to a structural component or functional component of said cell. Non-limiting examples of image features include brightness, regional brightness, intensity, regional intensity, contrast, embeddings, background intensity, density. Image feature extraction generally begins from an initial set of measured data and builds derived values (features) intended to be informative and non-redundant, facilitating the subsequent learning and generalization steps. Image feature extraction is related to dimensionality reduction.

As used herein, the term “genetic alteration” refers to a difference (e.g., an insertion, deletion, or substitution of one or more nucleotides) in a nucleic acid molecule relative to a wild type sequence. When present in the coding region of a nucleic acid, a genetic alteration may be “silent” (i.e., results in no phenotypic effect) or may alter the function of the expression product of the coding region. When a genetic alteration occurs to the regulatory region of a gene or operon, the genetic alteration may either have no effect or alter the expression characteristics of the regulated nucleic acid. A genetic alteration can mean the deletion of all or part of a gene. A genetic alteration can mean the insertion of an additional copy of a gene already present in the genome of the host cell, or insertion of a non-native gene.

As used herein, the term “modulation of expression” may refer to up-regulation of expression by, for example, a regulated or constitutive promoter inserted upstream of a gene, gene cloning in a multi-copy plasmid, an upstream or downstream regulatory sequence (e.g., a cis-acting regulatory sequence, such as an upstream activating sequence (UAS)), or another mechanism. The term “modulation of expression” may refer to down-regulation of expression by, for example, replacing a native promoter with a “weaker” promoter, complete gene inactivation or deletion, an upstream or downstream regulatory sequence, manipulation of the 3′-untranslated region (3′-UTR) of a gene), or another mechanism.

As used herein, the term “bioproduction” is intended to mean production of a compound (e.g., a terpene or isoprenoid) by way of biological or enzymatic synthesis (as opposed to chemical synthesis). In some implementations, bioproduction may be performed by a transgenic organism or microbe that has been engineered to express enzymes involved in the biological synthesis of a compound of interest.

As used herein, the term “strain” refers to microbial cells of a particular species which have common characteristics. In general, cells of a particular strain share the same genotype, and two cells that do not share the same genotype are considered to be of different strains. Unless indicated to the contrary, the terms “strain” and “cell” are used interchangeably herein. As one skilled in the art would recognize, microbial cell (e.g., yeast) strains are composed of individual microbial cells. Further, individual microbial cells have specific characteristics (e.g., a particular growth rate or level of target biomolecule production) which identifies them as being members of their particular strain.

As used herein, the term “biomolecule” or “biological molecule” refers to any of numerous substances produced by cells and living organisms. Biomolecules have a wide range of sizes and structures, and may perform a vast array of functions. Non-limiting examples of biomolecules include saccharides (e.g., monosaccharides, disaccharides, etc.), carbohydrates, fatty acids, lipids (e.g., glycolipids, phospholipids, sterols, etc.), nucleosides, nucleotides, nucleic acids (e.g., deoxyribonucleic acids (DNA), ribonucleic acids (RNA)), amino acids, peptides, polypeptides, proteins, vitamins, neurotransmitters, metabolites, and enzymes. Biomolecules may be endogenous, synthetic, or modified. A biomolecule can be or comprise a product of interest (i.e., a compound to be produced by a living organism, such as a cell (e.g., a microbial cell)). A product of interest can be or comprise, for example, a small molecule, enzyme, protein, peptide, amino acid, organic acid, synthetic compound, fuel, alcohol, primary extracellular metabolite, secondary extracellular metabolite, intracellular component molecule, and combinations thereof. A product of interest can be or comprise a terpene or a terpenoid. Non-limiting examples of products of interest include farnesene, ethanol, bakuchiol, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene. Additionally or alternatively, a product of interest may be a biomolecule, such as an amino acid, modified amino acid, protein, nucleic acid, lipid, or other molecule.

As used herein, the term “phenotype” refers to observable physical characteristics dependent upon the genetic constitution of a microorganism. Examples of phenotypes include, but are not limited to, the ability to express particular gene products and the ability to produce certain amounts of a particular compound in a specified amount of time, an observable stress response, cell size, growth rate, division rate, resistance to cellular stress, energy utilization, oxygen utilization, and signs of fitness.

As used herein, the term “over-produce” refers to the production of a biomolecule by a cell in an amount greater than the amount produced by a reference strain (e.g., a parent strain). One example of an over-producing strain is a strain generated from a parent strain (i.e., the reference strain) using mutagenesis or other genetic editing, which produces more of a particular target biomolecule than the parent. While the term “mutagenesis” is used in the present disclosure, it is used as an illustrative example of genetic editing, and may therefore encompass genetic editing in general. Thus, the strain generated by mutagenesis would “over-produce” the target biomolecule in comparison to the parent, reference strain.

As used herein, the term “attenuate” means to reduce the function of. For example, as used herein, an “attenuated gene” is a gene whose expression and/or function is reduced relative to that of a non-attenuated version of the gene. An attenuated gene is a gene may have an activity level that is less than 100% (e.g., 99%, 95%, 90%, 80%, 50%, 25%, 20%, 10%, 5%, or 0%) of the activity level of a non-attenuated version of the gene.

As used herein, the term “parent strain” refers to a strain of a microbial cell subjected to mutagenesis to generate a microbial cell with desired characteristics. Thus, use of the phrase “parent strain” does not necessarily equate with the phrase “wild type” or provide information about the history of the referred to strain.

As used herein, the terms “engineered microbial cell” refers to a modified microbial cell, such as a yeast cell, wherein the modification can be selected from e.g., increased expression of a gene, inhibited expression of a gene, introduction of new gene(s), introduction of mutant gene(s), or mutation/genetic alteration of gene(s), wherein the increased expression or inhibited expression of a gene can be achieved by using common techniques in the art, such as gene deletion, changed gene copy number, changed gene promoter (e.g. by using a strong or weak promoter), etc. In some implementations, an engineered microbial cell is a modified microbial cell capable of producing high levels of a compound or biomolecule of interest.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., microbial cell, bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the host cell), as compared to the naturally-occurring organism from which it was derived. It is understood that in some implementations, the terms refer not only to the particular recombinant host cell in question, but also to the progeny or potential progeny of such a host cell.

### Overview

Provided herein are methods for utilizing high content imaging (HCI) data to determine or predict the phenotype of a microbial cell. Determining the phenotype of a particular strain of a particular microbe or microbial cell (e.g., a particular transgenic strain of a microbial, e.g., a particular transgenic yeast strain), and the underlying biology driving that phenotype, are important to engineering strains with desired functions. A desired function may include but is not limit to the production of a product (e.g., a target biomolecule) at scale in a bioreactor. High throughput phenotyping, such as high throughput imaging, of cells can be used as a surrogate or a complement to direct phenotype measurements, such as but not limited to titer, fitness/growth, dysfunctional biology, response to chemical perturbations, and various omics measurements (e.g., transcriptomic measurements, proteomic measurements, etc.). In some implementations, high throughput phenotyping comprises imaging, such as high content imaging.

In general, HCI may be used to generate an image feature (e.g., a stain, density, shape, size, embedding, etc.) from high resolution images of cells with known phenotypes. The images can be created using bright field microscopy, fluorescence microscopy, or other types of microscopy. For example, images of cells labeled with fluorescent stains that are specific to certain cellular components such as DNA (nucleus) or the cell membrane can be obtained using a fluorescence-enabled high-resolution cell imaging system. A plurality of images of cells that represent some variation in biological state, such as between strains, gene knockouts, metabolic variance, or other variables that result in different phenotypes, can be used to create a training data set. The raw cell images can be processed to normalize signal (i.e. background subtraction, denoising, etc.) and then the image features can be extracted using one or more computational tools (e.g., convolutional neural networks (CNN)) to effectively convert the image into high dimensional vector space. Attention weight learning algorithms can be applied to help amplify signal over background. The image features can represent, for example, different cellular morphologies across the cells from which they were extracted, and can be used to model not just the phenotypes from which they were generated, but can also be used to model phenotypes that are not obviously related to the image features of the phenotypes (e.g. the phenotypes represented in the training data set).

The methods described herein achieve resource- and cost-effective identification and prediction of microbial cell phenotypes without the need to assay direct phenotypic outputs (e.g., titer or production of a target biomolecule, etc.). These approaches are useful for understanding microbial cell biology, and are especially useful in the biomanufacturing of biomolecules and other compounds that can be produced by a microbial cell (e.g., yeast).

The methods disclosed herein offer a high throughput, less expensive surrogate for direct phenotype measurements, such as transcriptomic, proteomic, and metabolomic measurements, which are generally performed only sparingly on strains of interest for bioproduction.

### Artificial Intelligence (AI) Models and Machine Learning

As referred to herein, AI and AI models can be used to describe a variety of different models that can be used in predicting states and other information associated with microbial cell phenotypes. In some implementations, an AI model can comprise machine learning (ML). In some implementations, an AI model can be or comprise a machine learning (ML) model. In some implementations, recurrent neural network (RNN) models are utilized for generating predictions. RNNs are a class of artificial neural networks where connections between nodes form a directed graph along a temporal sequence. More specifically, long short-term memory (LSTM) models may be utilized in generating predictions. LSTMs are a specific type of artificial RNN architecture that are used primarily for deep learning. LSTMs can classify and process entire sequences of time-series data and can make predictions based on said time-series data. Advantageously, LSTMs can account for lags of unknown duration between important events in a time series. In some implementations, other types of AI models such as convolutional neural networks (CNNs) are utilized in generating predictions. Accordingly, it should be appreciated that various types of AI models can be utilized in generating predictions described in this disclosure.

As described herein, the term “AI” can be used to refer to a variety of different types of AI models used for generating predictions, such as, for example, machine learning models. In particular, the systems and methods described herein may leverage neural networks to generate predictions associated with microbial cell phenotypes. For example, recurrent neural networks (RNNs) such as long short-term memory (LSTM) networks may be used to generate predictions. It should be appreciated that other types of neural networks and/or different AI models can be utilized in generating predictions associated with biological processes. For example, convolutional neural networks (CNNs), multi-layer perceptrons, etc. can be utilized in generating predictions. In some implementations, multiple AI models are trained for generating specific predictions and can be tested for an accuracy of predictions relative to a known data set. Based on the determined accuracy of each AI model, an AI model that generates predictions that most closely match the known data set can be selected and utilized.

In a non-limiting example, the AI model may utilize a deep neural network (DNN). A DNN is a branch of neural networks that comprises a stack of layers each performing a specific operation, e.g., convolution, pooling, loss calculation, etc. Each intermediate layer receives the output of the previous layer as its input. The beginning layer is an input layer, which is directly connected to or receives an input data structure that includes the data items in one or more machine-readable objects, and may have a number of neurons equal to the data items in one or more machine-readable objects provided as input.

As described in greater detail below, AI models utilized for generating predictions can be trained based on a variety of different data sources. In some implementations, the AI models are trained based on publicly available biological data, such as data obtained from a microbial cell image database or repository. In some implementations, the AI models are trained based on data generated based on a simulated environment. In such a case, the simulated environment can be structured to mimic a living biological system. Advantageously, using a simulation for generating training data may result in a larger set of training data being generated than may otherwise be generated by a living biological system. In some implementations, the AI models are trained based on newly acquired data. Newly acquired data may include data that are not publicly available. For example, newly acquired data may include bright field microscopy or fluorescence microscopy data obtained from imaging microbial cells or microbial cells of interest. In some implementations, the newly acquired data comprise data obtained from a plurality of different strains of microbial cells representing a plurality of microbial cell phenotypes. In some implementations, the AI models are trained based on a mix of training data. A mix of training data may include, for example, two or more of data generated by a simulation, data obtained from a database or repository, data gathered de novo from a living biological system, and/or from some other source of training data.

By utilizing AI models to generate predictions associated with microbial cell phenotypes, strains of microbial cells likely to harbor a particular phenotype can be identified or engineered for an intended purpose. For example, predicting the ability of a microbial cell to generate a particular compound of interest in a tank or bioreactor using AI models, can enable the identification and engineering of microbial cell strains likely to produce significant amounts of the compound of interest before ever subjecting a microbial cell to culturing in a tank or bioreactor.

In some embodiments, the AI model may be trained using a training dataset that has been procured specifically to indicate various attributes of microbial cell strains. For instance, the training dataset may include data associated with previously grown microbial cell strains and their corresponding HCIs and/or attributes. In a non-limiting example, the training dataset may include an HCI for a set of batches of microbial cell strains grown in a reactor. For each batch, a corresponding HCI may also be identified. In some embodiments, various attributes of the microbial cell strains, such as size, shape, surface geography, and the like may also be included within the training dataset. Additionally or alternatively, the HCI itself can be analyzed to identify these attributes.

After retrieving the data associated with previously known batches, various data grooming protocols may be used to pre-process the data. For instance, one or more processors or computer models may de-noise and/or de-duplicate the collected data. In some embodiments, outliers may be identified and removed, such that the AI model can be trained more efficiently.

The AI model may be trained using the training dataset via employing various machine learning techniques. Specifically, the AI model may be trained using a supervised, unsupervised, and/or semi-supervised learning method. In some embodiments, the amount of available data and whether labeling is available/feasible may dictate the type of machine learning techniques used.

In a non-limiting example of implementing a supervised learning technique, the AI model may use a labeled training dataset. In this learning technique, the training dataset may first be labeled. For instance, after the data is groomed, the training dataset can be labeled in accordance with known data. The labeling process can be performed manually and/or using a computer-implemented protocol(s), also known as automatic labeling or auto-labeling. Specifically, each dataset corresponding to a batch of a microbial cell strain, its attributes, and its HCI can be labeled as “fit” or “not fit.” Effectively, by labeling the data, the training dataset can be used as “ground truth” and used to train the AI model. Using various clustering methods and other machine learning techniques, the AI model may cluster the data and uncover patterns correlating the HCI of yeast, its attributes, and whether it was ultimately designated as “fit.”

When trained, the AI model may identify patterns that correlate certain visual attributes of an HCI that would likely give rise to the microbial cell strain being designated as “fit” or “unfit.” When trained, the AI model may be configured to ingest a new HCI and determine the likelihood of the corresponding microbial cell strain being designated as “fit.”

In a non-limiting example, the AI model may be trained using an unsupervised manner. In this example, the same steps as the supervised manner may be repeated. However, the training dataset may not be labeled using ground truth. The AI model may still ingest the training dataset and train itself accordingly. The AI model may infer the structures, patterns, and correlations present within the training dataset. The AI model may use various methods, such as image segmentation, image featurization, voxel identification, clustering, and/or density estimation techniques to identify the inherent structure of data without using explicitly provided labels, such as provided in the supervised learning method.

The training methods may not be limited to the above-described machine-learning techniques. For instance, in some embodiments, the AI model may be trained using a semi-supervised method.

During training, the analytics server may iteratively produce new predicted results. If the characteristics of the predicted result do not match the desired results (e.g., the real characteristics of yeast as designated within the training dataset), the AI model may iteratively revise one or more weight factors or internal parameters (e.g., parameters for different layers of a NN) and re-calculate the results. This iterative process can be repeated (the AI model continues the training) until and unless the computer-generated recommendation satisfies one or more accuracy metric thresholds and is within acceptable ranges.

In some configurations, the AI model may train itself using a predetermined portion (e.g., fold) of the ground-truth data. The AI model may then gauge its accuracy metrics (e.g., area under the curve, precision, and/or recall) using the remaining data points within the training dataset (e.g., second fold).

### Methods for Predicting a Microbial Cell Phenotype

Provided herein are methods for determining or predicting a phenotype of a microbial cell. In some implementations, a method for determining or predicting a phenotype of a microbial cell includes culturing the microbial cell, the phenotype of which is to be predicted. A microbial cell (e.g. a yeast cell) may be cultured using any technique known in the art. For example, a microbial cell may be cultured in a bioproduction reactor, fermentation tank, culture flask, or other suitable container for small-scale or large-scale bioproduction. One implementation of a container is a microplate. Various different culture media can be selected based on the particular species used and the growth conditions, among other things. In some implementations, minimal culture medium may be supplemented as needed to optimize growth and production of a given cell type (e.g., transgenic cell type).

In some implementations, a method for determining or predicting a phenotype of a microbial cell includes obtaining at least one high-content image of the microbial cell. A high-content image may be obtained using any imaging technique known in the art. In some implementations, a high-content image can be obtained using an imaging system (e.g., an optical microscopy system). Non-limiting examples of types of imaging systems include those utilizing bright field microscopy and/or fluorescence microscopy. In some preferred implementations, a high-content image can be obtained using fluorescence microscopy. For example, a high-content image of a microbial cell stained with one or more fluorescent labels (e.g., one or more antibodies comprising a fluorescent moiety, wherein the antibody specifically binds to a particular molecule, such as a specific protein, lipid, or DNA) can be obtained in accordance with the present disclosure. In some implementations, a high-content image of a microbial cell expressing one or more fluorophores may be obtained. The one or more fluorophores may be associated with a particular organelle, as in, for example, a fusion polypeptide comprising a fluorescently-tagged form of a protein normally expressed by the microbial cell.

In some implementations, a microbial cell is fixed with a fixing agent. Generally, a fixing agent is an agent that preserves cells or biological tissues from decay due to autolysis or putrefaction. As used herein, the term “to fix”-in reference to a cell, strain, or biological tissue-means to chemically preserve a cell, strain, or biological tissue from decay, such as decay due to autolysis or putrefaction. Such a process can be referred to as “chemical fixation.” Chemical fixation terminates any ongoing biochemical reactions occurring in the cells being fixed, and may also increase the cells' mechanical strength or stability. Any fixative or fixing agent can be used in accordance with the present disclosure. Non-limiting examples of fixing agents include crosslinking fixatives (e.g., formaldehyde, paraformaldehyde, or glutaraldehyde), precipitating fixatives (e.g., alcohols (e.g., ethanol, methanol), acetone, or acetic acid), oxidizing agents (e.g., osmium tetroxide, potassium dichromate, chromic acid, or potassium permanganate), mercurial, picrates, and HEPES-glutamic acid buffer-mediated organic solvent protection effect (HOPE) fixative. Other suitable agents may be employed.

The term “to fix”—in reference to a cell, strain, or biological tissue—can be used to describe a process of immobilizing a cell, strain, or biological tissue by allowing the cell, strain, or biological tissue to adhere to a surface. Such a process can be referred to as “physical fixation.” Accordingly, fixing a cell, strain, or biological tissue may comprise chemical fixation, physical fixation, or both.

In some implementations, fixing comprises adhering the microbial cells to a surface. In some implementations, adhering can include allowing cells to adhere to a surface passively, such as by allowing the cells to sink to a bottom surface of a container comprising cell culture medium, via gravity. In some implementations, adhering can include allowing cells to adhere to a surface via centrifugation. In some implementations, adhering can include allowing cells to adhere to a surface of a container containing the cells. A surface can be a surface of a container, such as a well, a culture dish, a flask, a plate (e.g., a microplate, a 96-well plate, a 384-well plate, etc.), bioreactor, or another vessel. To facilitate adherence to a surface, the surface can be coated with a solution or compound that can promote cell adherence. Non-limiting examples of solutions or compounds capable of promoting cell adherence include solutions or compounds comprising a polymeric protein or polypeptide, an extracellular matrix (ECM) protein (e.g., collagen Type I, fibronectin, or vitronectin), poly-L Lysine, Concanavalin A, or a combination thereof. In some implementations, a cell is adhered to a surface prior to staining the cell and/or imaging the cell.

In some implementations of a method for determining or predicting a phenotype of a microbial cell, a high-content image can be a single-channel image. A single-channel image can be, without limitation, a bright field image or a fluorescence image. In some implementations, a high-content image can be a multi-channel image obtained by imaging two or more (e.g., 2, 3, 4, 5 or more) different fluorophores. Non-limiting examples of fluorophores include fluorescent proteins (e.g., green fluorescent protein (GFP), red fluorescent protein (RFP), etc.), and fluorescent stains (e.g., 4′,6-diamidino-2-phenylindole (DAPI), which undergoes a 20-fold enhancement of fluorescence upon binding to AT regions of double stranded DNA (dsDNA). A fluorophore can be conjugated to a second molecule, such as an antibody or other ligand. Further examples of fluorophores include but are not limited to fluorescein isothiocyanate (FITC), rhodamine, Alexa Fluor™ 405, Alexa Fluor™ 488, Alexa Fluor™ 555, Alexa Fluor™ 610, Alexa Fluor™ 633, Alexa Fluor™ 647, Alexa Fluor™ 750, Texas Red (INVITROGEN), mCherry, Azurite, mKate-2, AMCA, coumarin, Cy5, Cy5.5, IR680, and Cy7.

In some implementations, a microbial cell is stained with at least one (e.g., at least 1, at least 2, at least 3, or more) staining agent. A staining agent can be, for example, a ligand, a binding agent, or a conjugate thereof. Any binding agent or ligand showing the desired specificity may be used. A binding agent or ligand can be an agent that specifically binds to a particular cellular or subcellular structure, organelle, biomolecule, or other cellular or subcellular component. For example, a binding agent or ligand can be an agent that binds to a cell structure component, such as actin filaments (e.g., F-actin) or microtubules (e.g., microtubule associated protein 2 (MAP2)). A binding agent or ligand can be a carbohydrate-binding protein, such as a lectin. Non-limiting examples of binding agents include Phalloidin and Concanavalin A (ConA). Other suitable agents may be employed. In some implementations, a conjugate of a binding agent or ligand is a fluorescent conjugate of the binding agent or the ligand.

In some implementations, a staining agent comprises at least one detection moiety, such as a fluorescent moiety. In some implementations, a staining agent comprises at least one binding moiety. A binding moiety can be, without limitation, a binding agent, a ligand, or an antibody or fragment thereof. In some implementations, a staining agent comprises at least one detection moiety and at least one binding moiety. Non-limiting examples of staining agent include rhodamine-conjugated Phalloidin (rhodamine-Phalloidin), Alexa Fluor™ Plus 555-conjugated Phalloidin (Alexa Fluor™ Plus 555 Phalloidin), fluorescein isothiocyanate (FITC)-conjugated Concanavalin A (fluorescein isothiocyanate (FITC) concanavalin A), and 4′,6-diamidino-2-phenylindole (DAPI). In some implementations of a method for determining or predicting a phenotype of a microbial cell, a high-content image may be a single-Z image. In some implementations, a high-content image can be a Z-stack image. In some implementations, a Z-stack image is a compilation of images obtained at multiple different image depths, without significantly varying the X- or Y-coordinates of the image. A Z-stack image may include two or more (e.g., 2, 3, 4, 5, 10, 20, or 50 or more) images obtained at multiple different image depths. In some preferred implementations, a Z-stack image comprising 5 images obtained at different image depths is acquired.

In some implementations of a method for determining or predicting a phenotype of a microbial cell, a high-content image is processed, such as by one or more image processing algorithms. In some implementations, raw images of microbial cells are processed to help normalize signal intensities, such as to subtract background intensity, and/or to enhance a signal-to-noise ratio. In some implementations, a single image from a Z-stack is selected for downstream processing. In some implementations, a single image from a Z-stack is selected for downstream processing, wherein the single image is the most in-focus image of the images in the Z-stack. A focus of a Z-stack image may be obtained using an image processing algorithm. Focus can be defined as the sum of the per channel variances of the pixel intensities. As the skewness of the pixel intensities can result in different relative weighting of each channel of a multi-channel image, a ‘Focus via Normalized Variance’ algorithm can be employed in order to find the focus. In some implementations, the ‘Rank Exponential Transform’ algorithm may be employed. The ‘Rank Exponential Transform’ algorithm is a pre-processing technique useful for reducing technical variation and batch effects across experimental conditions. Briefly, pixel intensities are ranked and mapped onto an exponential distribution function. Additionally, the transformed intensities are centered at 0. This transformation limits the effects experimental batches and technical noise, such as outliers or artifacts. The transformed pixel intensities are typically more consistent across different images per channel, as well as across channels per image. One or ordinary skill in the art is aware of various image processing algorithms, as well as their use and execution.

In some implementations of a method for determining or predicting a phenotype of a microbial cell, a high-content image is processed, said process comprising denoising. Denoising can be accomplished by, for example, applying a filter across an acquired or obtained Z-stack image. In some implementations, a 3D MinMedian filter is applied across a Z-stack image. The 3D MinMedian filter applies the median function across 3 adjacent Z-stacks, as well as a 3×3 convolution across the spatial dimensions. In some implementations, after applying a 3D MinMedian filter, the final intensity is the minimum of the original value and the 3D median filtered value. By computing the spearman correlation of adjacent Z-stacks per channel, the consistency of biological signal can be approximated. This technique makes use of the property that salient pixels should be more correlated/consistent in intensity across Z-stacks compared to background pixels.

In some implementations of a method for determining or predicting a phenotype of a microbial cell, a computer-based model that was trained with image features of known microbial cell phenotypes is employed. A computer-based model may be, without limitation, an AI model, a machine learning model, a deep learning model, or a logistic model. A computer-based model can be trained with various image features extracted from images of microbial cells of known microbial cell phenotypes. Image features that are useful for the disclosed methods can be developed using convolutional neural networks (CNN) to effectively convert an image into high dimensional vector space. Image features, in some implementations, represent different cellular morphologies across the cells from which they were extracted. In some implementations, image features can be used to model not only the phenotypes of the cells from which they were generated, but also phenotypes that are not obviously related to the phenotypes of the cells from which they were generated.

In some implementations of a method for determining or predicting a phenotype of a microbial cell, attention weight learning algorithms can be applied as a segmentation method to aid in amplifying signal intensity over background intensity in an image. In general, attention weights can be learned by a computer-based model as a by-product of attempting to predict a microbial cell phenotype. Briefly, areas of an image that deemed relevant are given high weights, and background signal is given low weight. When creating a representation of an image in a lower dimensional space, the attention weights can be used to focus this representation on relevant areas. Using the attention weights a single representation of an image that is focused on the cells can be created.

In some implementations, a method for determining or predicting a phenotype of a microbial cell comprises (a) culturing the microbial cell; (b) obtaining at least one high-content image of the microbial cell; (c) executing a computer-based model that was trained with selected image features of known microbial cell phenotypes; and (d) generating, with a processor, a determination or prediction of the phenotype of the microbial cell.

In some implementations, provided is a computer-implemented method for predicting a phenotype of a microbial cell. A computer-implemented method can be a method that utilizes a computer system. A computer-implemented method can be a method that is executed on a computer system. In some implementations, a computer system comprises a processor. In some implementations, a computer system comprises a non-transitory computer-readable medium (CRM) or memory. In some implementations, a computer-implemented method for predicting a phenotype of a microbial cell comprises populating a predictive machine learning model with a training data set. A training data set can include a plurality of images of microbial cells with known phenotypes. A training data set may include a plurality of image features extracted from a plurality of images of microbial cells with known phenotypes. Image features can be extracted from an image by, for example, using convolutional neural networks (CNN). In some implementations, a training dataset comprises at least one input variable representing at least one genetic alteration that has been introduced into a microbial cell. In some implementations, a training dataset comprises at least one measured phenotypic performance output variable representing at least one phenotypic measurement associated with an introduced genetic alteration. In some implementations, a training dataset comprises i) at least one input variable representing at least one genetic alteration that has been introduced into a microbial cell, and ii) at least one measured phenotypic performance output variable representing at least one phenotypic measurement associated with the introduced genetic alteration. In some implementations, the at least one phenotypic measurement comprises a high-content image feature related to the phenotypic measurement. In some implementations, a training dataset comprises i) at least one input variable representing at least one genetic alteration that has been introduced into a microbial cell, and ii) at least one measured phenotypic performance output variable representing at least one phenotypic measurement associated with the introduced genetic alteration, wherein the at least one phenotypic measurement comprises a high-content image feature related to the phenotypic measurement.

In some implementations, a computer-implemented method for predicting a phenotype of a microbial cell comprises generating, in silico, a pool of design candidate microbial cells. In some implementations, one or more of the design candidate microbial cells of the pool of design candidate microbial cells comprises at least one genetic alteration relative to a wild type microbial cell of the same strain. In some implementations, each design candidate microbial cell of the pool of design candidate microbial cells comprises at least one genetic alteration relative to a parent microbial cell of the same strain (i.e. a parent strain).

In some implementations, a computer-implemented method for predicting a phenotype of a microbial cell comprises utilizing a predictive machine learning model to predict an expected phenotypic measurement of members of a pool of design candidate microbial cells that comprise a combination of genetic alterations. In some implementations, a genetic alteration or a combination of genetic alterations are previously uncharacterized for improving phenotypic performance. A predicted expected phenotypic measurement can include, without limitation, titer, growth properties, stress response, omics data, and production of a product or compound of interest (e.g., a target biomolecule). In one implementation, stress response may include a physical response based on a hydrophobic or hydrophilic property of the cell.

In some implementations, a predictive machine learning model can be stored on and/or executed on a computer system. In some implementations, a predictive machine learning model is stored and executed on a computer system comprising a processor and a non-transitory computer-readable medium (CRM). In some implementations, the processor is coupled to the non-transitory CRM.

In some implementations, a computer-implemented method for predicting a phenotype of a microbial cell comprises: (a) populating a predictive machine learning model with a training data set, comprising: i) at least one input variable representing at least one genetic alteration that has been introduced into a microbial cell, and ii) at least one measured phenotypic performance output variable representing at least one phenotypic measurement associated with the introduced genetic alteration, wherein the at least one phenotypic measurement comprises a high-content image feature related to the phenotypic measurement; (b) generating, in silico, a pool of design candidate microbial cells incorporating the at least one genetic alteration; and (c) utilizing the predictive machine learning model to predict the expected phenotypic measurement of members of the pool of design candidate microbial cells that comprise a combination of genetic alterations selected from step (a) that are uncharacterized for improving phenotypic performance at the time of carrying out step (c); wherein the predicted expected phenotypic measurement is selected from the group consisting of titer, growth properties, omics data, and production of a product of interest.

Provided is a method for determining or predicting titer of a compound of interest by a microbial cell, comprising: executing a computer-based model to analyze at least one high content image of a microbial cell that produces a compound of interest, wherein the computer-based model was trained with image features associated with known microbial cell titers; and generating, with a processor, a determination or prediction of the titer of the compound of interest being produced by the microbial cell. In some implementations, the method further comprises executing a second computer-based model trained with image features associated with known microbial cell phenotypes to determine or predict a second phenotype selected from knock-out of a gene of interest, expression of a gene of interest, microbial fitness, a stress response, or a combination thereof.

Provided is a method for determining or predicting titer of a compound of interest by a microbial cell, comprising: executing a computer-based model to analyze at least one high content image of a microbial cell that produces a compound of interest, wherein the computer-based model was trained with image features associated with known microbial cell titers; and generating, with a processor, a determination or prediction of the titer of the compound of interest being produced by the microbial cell. In some implementations, the method further comprises executing a second computer-based model trained with image features associated with known microbial cell phenotypes to determine or predict a second phenotype selected from knock-out of a gene of interest, expression of a gene of interest, microbial fitness, a stress response, or a combination thereof.

Provided is a method for determining or predicting a phenotype of a microbial cell, comprising: executing a computer-based model to analyze at least one high content image of a microbial cell, wherein the computer-based model was trained with image features associated with known microbial cell phenotypes; and generating, with a processor, a determination or prediction of the phenotype of the microbial cell, wherein the known microbial cell phenotypes are selected from expression of a gene of interest, microbial fitness, a stress response, or a combination thereof.

It is to be appreciated that, in some implementations, the methods described herein can be performed on a microbial cell, a plurality of microbial cells, a strain, a microbial cell having a particular genotype, or a plurality of microbial cells sharing a particular genotype.

### Microbial Cells

Various prokaryotic and eukaryotic expression systems are commonly used for bioproduction, though factors including the growth conditions, type of fermenter utilized, toxicity (if any) of the product, and other metabolic considerations of the microbe producing the product of interest may be employed to select a suitable system. Thus, in some implementations, a host cell or a transgenic cell suitable for expressing a compound or product of interest may be a prokaryote. In in some implementations, a host cell or a transgenic cell suitable for expressing a compound or product of interest may be a eukaryote.

In some implementations, the isolated host cell or transgenic cell is a prokaryote. Model prokaryotic systems that may be utilized as a transgenic cell include but are not limited to Escherichia coli (E. coli), an Acinetobacter species, a Pseudomonas species, a Streptomyces species, and a Mycobacterium species. Additional suitable prokaryotic expression systems include, but are not limited to, Klebsiella, Lactococcus, Mannheimia, Corynebacterium, Vibrio, and Bacillis.

In some implementations, the isolated host cell or transgenic cell is a eukaryote. Model eukaryotic systems that may be utilized as a transgenic cell include but are not limited to yeast, such as Saccharomyces cerevisiae (S. cerevisiae), Pichia pastoris, or Kluyveromyces marxianus or other yeast species including other oleaginous yeast; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostelium discoideum. Additional suitable eukaryotic expression systems include, but are not limited to, Pichia pastoris, Yarrowia lipolytica, Kluyveromyces marxianus, Rhodosporidium toruloides. Aspergillus (oryzae, nidulans, niger), Trichoderma reesei, and Penicillium chrysogenum.

Various prokaryotic and eukaryotic expression systems can be utilized for the disclosed methods. In some implementations, the transgenic cell used in the methods may be a prokaryote, including but are not limited to Escherichia coli (E. coli), an Acinetobacter species, a Pseudomonas species, a Streptomyces species, a Bacillus species, and a Mycobacterium species. Additionally suitable prokaryotic expression systems include, but are not limited to, Klebsiella, Lactococcus, Mannheimia, Corynebacterium, Vibrio, and Bacillis. In some implementations, the transgenic cell used in the methods may be a eukaryote, including but are not limited to Saccharomyces cerevisiae (S. cerevisiae) or other yeast species; a filamentous fungi, optionally selected from an Aspergillus species and a Trichoderma species; an algae, optionally selected from Botryococcus braunii, Chlorella sp., Crypthecodinium cohnii, Cylindrotheca sp., Nitzschia sp., Phaeodactylum tricornutum, Schizochytrium sp., and Tetraselmis suecia; and an amoeba, which is optionally Dictyostelium discoideum. Additional suitable eukaryotic expression systems include, but are not limited to, Pichia pastoris, Yarrowia lipolytica, Kluyveromyces marxianus, Rhodosporidium toruloides. Aspergillus (oryzae, nidulans, niger), Trichoderma reesei, and Penicillium chrysogenum.

The disclosed methods can be carried out in a bioproduction reactor (i.e. “bioreactor”), fermentation tank (i.e. “tank”), culture flask, or other suitable containers for bioproduction. Various different culture media can be selected based on the particular transgenic species used and the growth conditions, among other things. In some implementations, minimal culture medium may be supplemented as needed to optimize growth and production of a given transgenic cell type. For example, in some implementations, such as those utilizing transgenic S. cerevisiae, the culture medium may comprise about 3% w/v maltodextrin, about 0.2% w/v glucose, alpha-amylase, or any combination thereof.

### Phenotypes of Microbial Cells

The methods provided herein can be used to predict a phenotype of a microbial cell. A phenotype is an observable characteristic or trait of an organism. Generally, a phenotype of an organism is a result of (1) the expression of the organism's genetic code (i.e., its genotype), and (2) the influence of environmental factors (e.g., growth conditions, etc.). In some implementations, a phenotype to be predicted by the methods provided herein may be related to, without limitation, cell health or viability, cell morphology, cell biochemical or physiological properties (i.e., expression and/or production of a particular biomolecule).

In some implementations, a phenotype is the production of a compound of interest by the microbial cell. A compound of interest can be any compound that the microbial cell is competent to produce. Non-limiting examples of compounds of interest include ethanol or an isoprenoid (e.g., a sesquiterpene, a monoterpene, a diterpene, or a meroterpene), including but not limited to bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, or valencene. A compound of interest can be a terpene or a terpenoid. In some implementations, a compound or product of interest can be or comprise, for example, a small molecule, enzyme, protein, peptide, amino acid, organic acid, synthetic compound, fuel, alcohol, primary extracellular metabolite, secondary extracellular metabolite, intracellular component molecule, and combinations thereof.

Provided herein are methods for determining or predicting titer of a compound of interest by a microbial cell, comprising: executing a computer-based model to analyze at least one high content image of a microbial cell that produces a compound of interest, wherein the computer-based model is trained with image features associated with known microbial cell titers; and generating, with a processor, a determination or prediction of the titer of the compound of interest being produced by the microbial cell.

### Methods for Engineering Microbial Cells

Provided herein are methods for engineering a microbial cell to have a desired phenotype. In some implementations, a method of engineering a microbial cell to have a desired phenotype comprises generating at least one design candidate microbial cell incorporating at least one genetic feature associated with a desired phenotype. A design candidate microbial cell can be generated in silico or in vivo. For example, design candidate microbial cell can be generated using known genetic pathway information (e.g., known effects of genetic manipulation (e.g., gene knockout) on a particular microbial cell phenotype). In silico methods for generating a design candidate cell may include the use of a computer, a computer system, or a computer simulation. In silico methods for the design of transgenic cells are well known in the art. In vivo methods for design and optimization of a microbial cell can include genetically modifying a microbial cell (e.g., yeast). Genetic modification may include, for example, transformation of the cell with one of more nucleic acids that encode a gene or genes of interest (e.g., heterologous or native genes); overexpression, knockdown or knockout of a gene or genes of interest; or other forms of genetic alteration.

In some implementations, a method of engineering a microbial cell to have a desired phenotype comprises constructing a design candidate microbial cell. Engineered microbial cells of the present disclosure, including design candidate microbial cells, may be constructed by any of the methods and techniques known and available to those skilled in the art. Illustrative examples of suitable methods for constructing microbial cells include gene integration techniques (e.g., mediated by transforming linear DNA fragments and homologous recombination) and transduction mediated by the bacteriophage Pl. These methods are well known in the art.

In some implementations, a method of engineering a microbial cell to have a desired phenotype comprises culturing a design candidate microbial cell. A microbial cell (e.g. a yeast cell) may be cultured using any technique known in the art. For example, a microbial cell may be cultured in a multi-well plate (e.g., 96-well plate, 384-well plate, etc.), bioproduction reactor, fermentation tank, culture flask, culture dish, or other suitable container for small-scale or large-scale bioproduction. Various different culture media can be selected based on the particular species used and the growth conditions, among other things. In some implementations, minimal culture medium may be supplemented as needed to optimize growth and production of a given cell type (e.g., transgenic cell type).

In some implementations, a method of engineering a microbial cell to have a desired phenotype comprises determining the phenotype of the at least one design candidate microbial cell using a high-content imaging (HCI)-based model. In some implementations, determining the phenotype of the at least one design candidate microbial cell comprises (i) obtaining at least one high-content image of the at least one design candidate microbial cell; (ii) executing a computer-based model that was trained with image features associated with at least one phenotypic measure; and (iii) generating with a processor a prediction of the phenotype of the microbial cell. In some implementations, the HCI-based model can be or can have been trained with a data set, comprising: i) at least one input variable representing at least one genetic feature, and ii) at least one measured phenotypic performance output variable representing at least one phenotypic measurement associated with the genetic feature. In some implementations, the at least one phenotypic measurement corresponds to an HCI image feature. Generally, a genetic feature refers to a genetic alteration or lack thereof.

In some implementations, a method of engineering a microbial cell to have a desired phenotype comprises (a) generating, in silico, at least one design candidate microbial cell incorporating at least one genetic feature associated with a desired phenotype; (b) engineering the at least one design candidate microbial cell; (c) culturing the at least one design candidate microbial cell; and (d) determining the phenotype of the at least one design candidate microbial cell using a high-content imaging (HCI)-based model.

### Apparatus for Microbial Cell Phenotype Monitoring

Provided herein is an apparatus or system for the monitoring of a microbial cell phenotype. In some implementations, an apparatus for the monitoring of a microbial cell phenotype comprises a bioreactor. In some implementations, an apparatus for the monitoring of a microbial cell phenotype comprises a fermentation system. In some implementations, a bioreactor or fermentation monitoring system comprises (a) a tank for culturing a population of microbial cells (b) a camera capable of obtaining high content images of the population of microbial cells in the tank, and (c) a processing system connected to the camera such that the high content images obtained by the camera can be used to predict a phenotype or function of individual cells within the population of microbial cells while the population of cells is being cultured. In one implementation, “population” may refer to a group of cells of a single given strain, or of different strains.

### EXAMPLES

These examples are provided for illustrative purposes only and not to limit the scope of the claims provided herein.

### Example 1. Experimental Workflow 1

The present example illustrates an exemplary experimental workflow employing high-content imaging (HCI) of yeast cells in order to predict a yeast cell phenotype.

### Sample Preparation and High-Content Imaging

Yeast cells were cultured in a bioreactor. Following yeast culture, cells were transferred to a multi-well plate and fixed for 30 minutes with 3.7% formaldehyde in cell medium in a 384 well MTP. Cells were then washed and fixed with 4% formaldehyde in phosphate buffered saline (PBS). Cells were then stained for multiple cellular markers. Stain 1 was actin with rhodamine-phalloidin. Stain 2 was fluorescein isothiocyanate (FITC) Concanavalin A. Following staining, cells were washed two times before mounting with a mounting buffer containing 4′,6-diamidino-2-phenylindole (DAPI) to stain nuclear DNA. Following mounting, the plate containing the mounted cells was sealed, and the cells were subjected to high-content imaging (HCI) on a microscope/HCI imager. Each well yielded multi-channel Z-stack images of cells, each Z-stack containing 5 images of the same field of cells at varying image depths.

After the high-content images of cells were obtained, the images were processed to reduce background fluorescence signal and other noise artifacts. The ‘Focus via Normalized Variance’ algorithm was employed in order to find the focus of the Z-stack. The ‘Rank Exponential Transform’ algorithm was employed to reduce technical variation and batch effects across experimental conditions.

### Phenotype Prediction Model Training and Execution

The results of this study showed that biological information regarding the phenotype of a microbial cell—in this case yeast—was gleaned from images, and this information can be of broad utility in modeling various phenotypes as well as tying phenotypes to particular biological function. This suggests that various image features can be generated on a particular set of cells, and then models can be trained and re-trained routinely to assess phenotypic characteristics such as titer, growth properties, omics data, etc.

### Example 2. Experimental Workflow 2

The present example illustrates an exemplary experimental workflow employing high-content imaging (HCI) of yeast cells in order to predict a yeast cell phenotype.

### Sample Preparation and High-Content Imaging

Yeast cells were cultured in a 96 well microplate. Following yeast culture, cells were transferred to a Concanavalin coated 384 well imaging plate, adhered via centrifugation and fixed for 30 minutes with 3.7% formaldehyde. After 30 minutes of fixing the cells were washed in phosphate buffered saline (PBS). Cells were then stained for multiple cellular markers. Stain 1 was Alexa Fluor™ Plus 555 Phalloidin to stain actin filaments. Stain 2 was fluorescein isothiocyanate (FITC) Concanavalin A to stain the cell wall. Stain 3 was 4′,6-diamidino-2-phenylindole (DAPI) to stain nuclear DNA. Following staining, the plate containing the stained cells was washed and filled with phosphate buffered saline (PBS), sealed, and the cells were subjected to high-content imaging (HCI) on a microscope/HCI imager. Each well yielded multi-channel Z-stack images of cells, each Z-stack containing 5 images of the same field of cells at varying image depths.

After the high-content images of cells were obtained, the images were processed to reduce background fluorescence signal and other noise artifacts.] After denoising the ‘Focus via Normalized Variance’ algorithm was employed in order to find the focus of the Z-stack. The ‘Rank Exponential Transform’ algorithm was employed to reduce technical variation and batch effects across experimental conditions.

### Phenotype Prediction Model Training and Execution

Machine learning models were trained on numerous datasets, including a gene knockout (KO) dataset, a stressor dataset, and titer prediction (Referencel and Reverence2) datasets.

### Results

The results of this study showed that biological information regarding the phenotype of a microbial cell—in this case yeast—was gleaned from images, and this information can be of broad utility in modeling various phenotypes as well as tying phenotypes to particular biological function. This suggests that various image features can be generated on a particular set of cells, and then models can be trained and re-trained routinely to assess phenotypic characteristics such as titer, growth properties, omics data, etc.

### Example 3. Predicting Gene Knockouts with Deep Learning Models

The present example illustrates that gene knockouts in yeast can be predicted using HCI deep learning models.

The experiment included 44 distinct “treatment” conditions, comprising 42 distinct gene knockouts introduced to yeast strain STR063, a parent STR063 as a control, and CEN.PK yeast strain as a wild type control. Images of cells from each of these groups were used to train a deep learning model for the purpose of generating image features, which were then be further analyzed in the context of the experiment, and other experiments.

### Test Data Accuracy

Henceforth in this example, all metrics only relate to the test dataset of sites within a particular plate. Below is a table of the top k accuracy metrics. These data suggest the model was able to generalize to previously unseen images.

TABLE 1

| | |
|---|---|
| Top 1 Accuracy | 0.51 |
| Top 2 Accuracy | 0.628 |
| Top 3 Accuracy | 0.682 |
| Top 4 Accuracy | 0.723 |
| Top 5 Accuracy | 0.758 |

### Comparison of Treatment- and Pathway-Level Accuracy

The following section includes accuracy metrics at the treatment- and pathway-level. In order to compare within pathways, the treatment-level pathway accuracy was calculated as the average correctness treatment-level predictions within a pathway. For the pathway-level accuracy, the predictions of all treatments within a pathway were summed to determine the pathway prediction. The pathway accuracy was 0.577, and the Treatment Accuracy was 0.510.

TABLE 2

| | | Parent | | | |
|---|---|---|---|---|---|
| | Entity | Strain | Integrated Stitches | gene_name | pathway |
| 0 | STR168 | STR063 | ADH2\hat{ }::pTEF1 > KanR- | ADH2 | ethanol |
| | | | tHUG1 (A3) | | degradation |
| 1 | STR169 | STR063 | ADH3\hat{ }::pTEF1 > KanR- | ADH3 | ethanol |
| | | | tHUG1 (A4) | | degradation |
| 2 | STR170 | STR063 | ADH4\hat{ }::pTEF1 > KanR- | ADH4 | ethanol |
| | | | tHUG1 (A5) | | degradation |
| 3 | STR171 | STR063 | ADH5\hat{ }::pTEF1 > KanR- | ADH5 | ethanol |
| | | | tHUG1 (A6) | | degradation |
| 4 | STR172 | STR063 | ALD2\hat{ }::pTEF1 > KanR- | ALD2 | ethanol |
| | | | tHUG1 (H2) | | degradation |
| 5 | STR173 | STR063 | ARA1\hat{ }::pTEF1 > KanR- | ARA1 | dehydro-D- |
| | | | tHUG1 (A9) | | arabinono-1,4- |
| | | | | | lactone |
| | | | | | biosynthesis |
| 6 | STR174 | STR063 | BAT2\hat{ }::pTEF1 > KanR- | BAT2 | methionine |
| | | | tHUG1 (A11) | | salvage pathway |
| 7 | STR175 | STR063 | CAR1\hat{ }::pTEF1 > KanR- | CAR1 | arginine |
| | | | tHUG1 (B5) | | degradation |
| | | | | | (anaerobic) |
| 8 | STR176 | STR063 | CDH1\hat{ }::pTEF1 > KanR- | CDH1 | Cell cycle |
| | | | tHUG1 (B7) | | |
| 9 | STR177 | STR063 | CPT1\hat{ }::pTEF1 > KanR- | CPT1 | phospholipid |
| | | | tHUG1 (H3) | | biosynthesis |
| | | | | | (Kennedy |
| | | | | | pathway) |
| 10 | STR178 | STR063 | DDC1\hat{ }::pTEF1 > KanR- | DDC1 | Cell cycle |
| | | | tHUG1 (C1) | | |
| 11 | STR179 | STR063 | DPH1\hat{ }::pTEF1 > KanR- | DPH1 | diphthamide |
| | | | tHUG1 (C3) | | biosynthesis |
| 12 | STR180 | STR063 | DPH5\hat{ }::pTEF1 > KanR- | DPH5 | diphthamide |
| | | | tHUG1 (C4) | | biosynthesis |
| 13 | STR181 | STR063 | DUN1\hat{ }::pTEF1 > KanR- | DUN1 | Cell cycle |
| | | | tHUG1 (C5) | | |
| 14 | STR182 | STR063 | EST3\hat{ }::pTEF1 > KanR- | EST3 | Chrom Org |
| | | | tHUG1 (H4) | | |
| 15 | STR183 | STR063 | ETR1\hat{ }::pTEF1 > KanR- | ETR1 | Metabolism |
| | | | tHUG1 (C6) | | |
| 16 | STR184 | STR063 | FAA1\hat{ }::pTEF1 > KanR- | FAA1 | fatty acid |
| | | | tHUG1 (C7) | | oxidation |
| | | | | | pathway |
| 17 | STR185 | STR063 | FBP1\hat{ }::pTEF1 > KanR- | FBP1 | gluconeogenesis |
| | | | tHUG1 (C9) | | |
| 18 | STR186 | STR063 | FOX2\hat{ }::pTEF1 > KanR- | FOX2 | fatty acid |
| | | | tHUG1 (C10) | | oxidation |
| | | | | | pathway |
| 19 | STR187 | STR063 | GLG2\hat{ }::pTEF1 > KanR- | GLG2 | glycogen |
| | | | tHUG1 (C12) | | biosynthesis |
| 20 | STR188 | STR063 | GPD1\hat{ }::pTEF1 > KanR- | GPD1 | glycerol |
| | | | tHUG1 (D2) | | biosynthesis |
| 21 | STR189 | STR063 | GPP2\hat{ }::pTEF1 > KanR- | GPP2 | glycerol |
| | | | tHUG1 (D6) | | biosynthesis |
| 22 | STR190 | STR063 | GUT2\hat{ }::pTEF1 > KanR- | GUT2 | glycerol |
| | | | tHUG1 (D4) | | degradation |
| 23 | STR191 | STR063 | HCM1\hat{ }::pTEF1 > KanR- | HCM1 | Transcription |
| | | | tHUG1 (D5) | | |
| 24 | STR192 | STR063 | ISA2\hat{ }::pTEF1 > KanR- | ILV2 | acetoin |
| | | | tHUG1 (D11) | | biosynthesis |
| 25 | STR193 | STR063 | KAR3\hat{ }::pTEF1 > KanR- | KAR3 | Chrom seg |
| | | | tHUG1 (E1) | | |
| 26 | STR194 | STR063 | LAC1\hat{ }::pTEF1 > KanR- | LAC1 | sphingolipid |
| | | | tHUG1 (E3) | | metabolism |
| 27 | STR195 | STR063 | LCB3\hat{ }::pTEF1 > KanR- | LCB3 | sphingolipid |
| | | | tHUG1 (E4) | | metabolism |
| 28 | STR196 | STR063 | LGE1\hat{ }::pTEF1 > KanR- | LGE1 | Chrom Org |
| | | | tHUG1(E5) | | |
| 29 | STR197 | STR063 | NMA1\hat{ }::pTEF1 > KanR- | NMA1 | NAD salvage |
| | | | tHUG1 (E6) | | pathway |
| 30 | STR198 | STR063 | PDA1\hat{ }::pTEF1 > KanR- | PDA1 | pyruvate |
| | | | tHUG1 (E9) | | dehydrogenase |
| | | | | | complex |
| 31 | STR199 | STR063 | POL32\hat{ }::pTEF1 > KanR- | POL32 | DNA replication |
| | | | tHUG1 (F1) | | |
| 32 | STR200 | STR063 | POT1\hat{ }::pTEF1 > KanR- | POT1 | fatty acid |
| | | | tHUG1 (F2) | | oxidation |
| | | | | | pathway |
| 33 | STR201 | STR063 | POX1\hat{ }::pTEF1 > KanR- | POX1 | fatty acid |
| | | | tHUG1 (G12) | | oxidation |
| | | | | | pathway |
| 34 | STR202 | STR063 | PYC1\hat{ }::pTEF1 > KanR- | PYC1 | TCA cycle, |
| | | | tHUG1 (H5) | | aerobic |
| | | | | | respiration |
| 35 | STR203 | STR063 | SDH1\hat{ }::pTEF1 > KanR- | SDH1 | aerobic |
| | | | tHUG1 (F7) | | respiration, |
| | | | | | electron transport |
| | | | | | chain |
| 36 | STR204 | STR063 | SDH2\hat{ }::pTEF1 > KanR- | SDH2 | aerobic |
| | | | tHUG1 (F8) | | respiration, |
| | | | | | electron transport |
| | | | | | chain |
| 37 | STR205 | STR063 | TKL1\hat{ }::pTEF1 > KanR- | TKL1 | non-oxidative |
| | | | tHUG1 (G4) | | branch of the |
| | | | | | pentose phosphate |
| | | | | | . . . |
| 38 | STR206 | STR063 | UBR1\hat{ }::pTEF1 > KanR- | UBR1 | Protein Metab |
| | | | tHUG1 (G5) | | |
| 39 | STR207 | STR063 | VPS64\hat{ }::pTEF1 > KanR- | VPS64 | Transport |
| | | | tHUG1 (G7) | | |
| 40 | STR208 | STR063 | YBP2\hat{ }::pTEF1 > KanR- | YBP2 | Ox stress |
| | | | tHUG1 (G8) | | |
| 41 | STR063 | STR063 | STR063 | STR063 | STR063 |
| 42 | STR283 | STR283 | STR283 | STR283 | STR283 |

### Per Class Performance Metrics

The predictive performance was highly heterogeneous, depending on the particular gene knockout. For example, STR063 (class 41) and Wildtype (class 42) did not have high accuracy scores, potentially indicating confusion with other ineffective gene knockouts.

### CONCLUSION

This deep learning model (trained on the correct label set) was able to predict gene knockout at an accuracy of 0.51. This analysis also shows that prediction accuracy was improved in certain pathways when gene knockouts were grouped as such. In aggregate, the accuracy of pathway-level prediction was 0.57, although this metric also benefitted from the reduced number of classes at the pathway-level.

### Example 4. Analysis of Deep Learning Model Image Features, Plate Titer, and In-Tank Tired Data

The present example illustrates that HCI model image features and plate titer data can be used to better predict in-tank titer.

### Plate Titer Data

The plate model of farnesene production consists of 4 replicate plates with a robust statistical design. These data will be aggregated at the strain level and used for a stand-alone analysis with the in tank data as well as incorporated into a predictive model using both plate titer and image feature data.

### Hold Out Test Plate Regression

For the purposes of understanding the consistency of the HCI data, the image feature were used to predict in-tank titer of strains in a hold out test plate. This section analyzes the four different plates from the titer prediction HCI dataset using each as a hold out test plate.

### Coefficients of Elastic Net Model

Each of the four models had slightly different image feature dimensions that were used to predict titer. Plate titer (index 0) and attention weights (a proxy for cell count, index 2) were used in every model, plate OD (index 1) was not used in any model. There were 6 other image features dimensions that are used across the 4 hold out plates to predict in-tank titer.

On each hold out plate test dataset, it was observed that plate titer with the addition of image features in a regression setting consistently improved the correlation with in-tank titer for each hold out test plate.

One benefit of the HCI data is the high degree of replication. The large number of observations at the well level allowed for better understanding of the variability in the HCI data and eventually led to more robust ensemble predictions.

Studies were also undertaken to assess how many replicates were needed to accurately predict the in-tank titer of a given strain. In non-control strains, the plate design had 8 wells per strain per plate (two source plate replicates×4 well replicates per source well). The 95% prediction intervals for the plate- and experiment-level predictions (consisting of 32=8 replicates×4 plates per experiment). While each strain varies, the model suggested that a reasonable 4 plate prediction interval within a few hundred in terms of titer.

### Conclusion

This example shows that HCI data can inform the strain selection process. These indicated that HCI image features were informative in predicting in-tank titer levels. In addition, prediction of in-tank titer values were consistent across plates as demonstrated by the hold out test plate regression analysis. In combination with plate titer values, the HCI image features were, and can be used to make predictions on the same strains with high correlation with real in-tank titer values.

These examples are provided for illustrative purposes only and not to limit the scope of the claims provided herein.

While certain implementations have been illustrated and described, it should be understood that changes and modifications can be made therein in accordance with ordinary skill in the art without departing from the technology in its broader aspects as defined in the following claims.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A method for determining or predicting a phenotype of a microbial cell, comprising:
(a) obtaining at least one high-content image of the microbial cell;
(b) executing a computer-based model that was trained with image features of known microbial cell phenotypes; and
(c) generating, with a processor, a determination or prediction of the phenotype of the microbial cell.

2. The method of claim 1, wherein the microbial cell is a yeast cell.

3. The method of claim 1, wherein the known microbial cell phenotypes associated with the image feature are selected from titer of a compound of interest that is produced by the microbial cell, knock-out of a gene of interest, expression of a gene of interest, microbial fitness, a stress response, or a combination thereof.

4. The method of claim 1, wherein the computer-based model is a deep learning model or a logistic model.

5. The method of claim 1, wherein the at least one high-content image is a fluorescent microscopy image.

6-7. (canceled)

8. The method of claim 1, further comprising, before obtaining at least one high-content image of the microbial cell, fixing the microbial cell with a fixing agent.

9-26. (canceled)

27. A method of engineering a microbial cell to have a desired phenotype, comprising:
(a) generating, in silico, at least one design candidate microbial cell incorporating at least one genetic feature associated with a desired phenotype;
(b) engineering the at least one design candidate microbial cell;
(c) culturing the at least one design candidate microbial cell; and
(d) determining the phenotype of the at least one design candidate microbial cell using a high-content imaging (HCI)-based model.

28. The method of claim 27, wherein determining the phenotype of the at least one design candidate microbial cell comprises
(i) obtaining at least one high-content image of the at least one design candidate microbial cell;
(ii) executing a computer-based model that was trained with image features associated with the at least one phenotypic measure; and
(iv) generating with a processor a prediction of the phenotype of the microbial cell.

29. The method of claim 27, wherein the HCI-based model is trained with a data set, comprising: i) at least one input variable representing the at least one genetic feature, and ii) at least one measured phenotypic performance output variable representing at least one phenotypic measurement associated with the genetic feature, wherein the at least one phenotypic measurement corresponds to a HCI image feature.

30-31. (canceled)

32. A bioreactor or fermentation monitoring system, comprising:
(a) a tank for culturing a population of microbial cells,
(b) a camera for obtaining high content images of the population of microbial cells in the tank,
(c) a processing system connected to the camera such that the high content images obtained by the camera are used to predict a phenotype or function of individual cells within the population of microbial cells while the population of cells is being cultured.

33. The method of claim 1, further comprising:
(a) populating the computer-based model with a training data set, comprising: i) at least one input variable representing at least one genetic alteration that has been introduced into the microbial cell, and ii) at least one measured phenotypic performance output variable representing at least one phenotypic measurement associated with the introduced genetic alteration, wherein the at least one phenotypic measurement comprises a high-content image (HCI) image feature related to the phenotypic measurement;
(b) generating, in silico, a pool of design candidate microbial cells incorporating the at least one genetic alteration; and
(c) utilizing the computer-based model to predict the expected phenotypic measurement of members of the pool of design candidate microbial cells that comprise a combination of genetic alterations selected from (a) that are uncharacterized for phenotypic performance at the time of carrying out (c);
wherein the predicted expected phenotypic measurement is selected from titer, growth properties, omics data, and production of a product of interest.

34. The method of claim 33, wherein the product of interest is selected from: a small molecule, an enzyme, a protein, a peptide, an amino acid, an organic acid, a synthetic compound, a fuel, alcohol, a primary extracellular metabolite, a secondary extracellular metabolite, an intracellular component molecule, and combinations thereof.

35-61. (canceled)

62. The method of claim 1, wherein the phenotype to be determined or predicted is titer of a compound of interest.

63. The method of claim 62, wherein the compound of interest is a terpene or terpenoid.

64. The method of claim 62, wherein the compound of interest is selected from bakuchiol, farnesene, farnesol, geosmin, geraniol, terpineol, limonene, myrcene, linalool, hinokitiol, pinene, cafestol, kahweol, cembrene, taxadiene, α-bisabolol, α-guaiene, bergamontene, and valencene.

65. The method of claim 1, further comprising executing a second computer-based model trained with image features associated with known microbial cell phenotypes to determine or predict a second phenotype selected from knock-out of a gene of interest, expression of a gene of interest, microbial fitness, a stress response, or a combination thereof.

66. The method of claim 1, wherein the microbial cell is prokaryotic.

67. The method of claim 66, wherein the microbial cell is selected from Escherichia coli (E. coli), an Acinetobacter species, a Pseudomonas species, a Streptomyces species, a Bacillus species, and a Mycobacterium species.

68. The method of claim 1, wherein the microbial cell is eukaryotic.

69. The method of claim 68, wherein the microbial cell is selected from a yeast, a filamentous fungus, an alga, and an amoeba.
